(19) United States
(12) Patent Application Publication
(10) Pub. No.: US 2026/0020768 A1
(43) Pub. Date: Jan. 22, 2026

(54) WEARABLE SENSING APPARATUS AND DIAGNOSTIC APPARATUS USING SAME

(71) Applicant: ZENTRY CO., LTD., Cheonan-si, Chungcheongnam-do (KR)

(72) Inventor: Hyunho CHANG, Cheonan-si, Chungcheongnam-do (KR)

(21) Appl. No.: 18/843,680

(22) PCT Filed: Jul. 4, 2022
(86) PCT No.: PCT/KR2022/009601

§ 371 Date: Mar. 21, 2025

(30) Foreign Application Priority Data
Mar. 3, 2022 (KR) 10-2022-0027433
Mar. 29, 2022 (KR) 10-2022-0038502

(51) Int. Cl. A61B 5/0205; A61B 5/00; A61B 5/024; A61B 5/08; A61B 5/282; A61B 7/04

(52) U.S. Cl. CPC A61B 5/0205; A61B 5/024; A61B 5/0803; A61B 5/0816; A61B 5/282; A61B 5/6831; A61B 5/7271; A61B 7/04; A61B 2560/0462; A61B 2562/0204; A61B 2562/0261

(57) ABSTRACT

The present invention relates to a wearable sensing apparatus and a diagnostic apparatus using same, the wearable sensing apparatus allowing measurement of: the breathing rate by means of the varying sensing values of the tensile force on a strap in response to the breathing of a test subject; and the heartbeat rate by means of the sound of the heart beats that is amplified by a diaphragm. The present invention may comprise: a main body housing provided with a first coupling member and a second coupling member, and having a pressure hole on the bottom surface; a strain sensor that measures the change in state of the first coupling member or the change in the contact point thereof; and a circuit substrate having an auditory sensor mounted thereon to measure the sound of the heat beats of a test subject amplified by a diaphragm.

### TECHNICAL FIELD

The present invention relates to a wearable sensing apparatus, and more specifically, to a wearable sensing apparatus capable of measuring the breathing rate and heartbeat rate of a subject, and a diagnosis apparatus using the same.

### BACKGROUND ART

Recently, as interest in health is increasing, researches on healthcare using electronic devices are actively conducted. For example, sensors mounted on an electronic device may collect information related to the electronic device, the outside of the electronic device, or the user, and it is most important for the user to continuously measure bio-signals in order to check his or her own condition. In relation to this, as techniques capable of monitoring exercise states or abnormal states of a user are required, electronic devices that provide a function of checking bio-signals of a user are developed.

In particular, among the bio-signals collected from the user, the breathing rate is one of vital signs for grasping the most basic vitality of the body, and various methods are used to measure the respiratory rate, i.e., the breathing rate per minute.

For example, the methods for measuring the respiratory rate or the breathing rate of a subject include as spirometry and capnometry.

The spirometry is a method of measuring the flow of air flowing in and out of the lungs using a spirometer, and the capnometry is a method of measuring CO₂ according to breathing. Although accuracy of these conventional methods is relatively high, the methods have limitations in that additional equipment is required and continuous monitoring is difficult.

Heart sounds are sounds generated when the heart contracts and expands. Although it is general that the heart sounds may be confirmed using a stethoscope, when confirming the heart sounds using a stethoscope, other noises are collected as well, there should be a filter to remove the noises.

In addition, when various devices are embedded in the stethoscope, the internal structure of the stethoscope is complex, and this is disadvantageous from the aspect of maintenance due to frequent failures.

In addition, since animals such as dogs (including puppies), cats, and birds may have a change in the breathing rate and heartbeat rate according to the external temperature although abnormal symptoms are not revealed, it is important to determine whether an increase in the breathing rate or heartbeat rate of a subject is due to an abnormal state (e.g., disease) or due to the external temperature. However, since conventional measurement devices do not consider the external temperature at all, when the breathing rate or heartbeat rate increases due to the influence of air temperature in an environment where the temperature outside the subject is higher than a reference value or lower than a reference value, an error of determining that the subject is in an abnormal state although the subject is in a normal state may occur.

### DISCLOSURE OF INVENTION

### Technical Problem

Therefore, the present invention has been made in view of the above problems, and it is an object of the present invention to provide a wearable sensing apparatus capable of measuring the breathing rate of a subject through a sensing value that changes through the tensile force of a strap according to breathing of the subject, and measuring the heartbeat rate of the subject through heartbeat sounds amplified through a diaphragm, and a diagnosis apparatus using the same.

### Technical Solution

To accomplish the above object, according to one aspect of the present invention, there is provided a wearable sensing apparatus comprising: a main body housing provided with a first coupling member and a second coupling member for coupling a strap on opposing sides, and having a pressing hole formed on a bottom surface; a strain sensor attached to the first coupling member or an inside of a first side wall of the main body housing provided with the first coupling member, and measuring a change in a shape of the first coupling member or an attached point of the first coupling member generated by tension of the strap according to breathing of the subject; a diaphragm coupled to the pressing hole and provided with a flexible plate; and a circuit board embedded in the main body housing, and provided with a stethoscope sensor that measures a heartbeat sound of the subject amplified by the diaphragm.

The first coupling member may be configured to include: an elastic body to which the strain sensor is attached on one side; and a coupling ring to which the strap is coupled, of which one side is connected to the other side of the elastic body, and the other side is separated from the elastic body.

A mounting groove ay be formed on a first side wall of the main body housing, and the first coupling member may be mounted on the main body housing through the mounting groove, wherein one side of the coupling ring coupled to the elastic body may be inserted into the mounting groove.

A bottom plate having a first sensing hole formed thereon may be further disposed at a portion on the bottom of the main body housing, the stethoscope sensor may be installed at a position corresponding to the first sensing hole on the circuit board installed in the main body housing, the heartbeat sound transferred by deformation of the diaphragm may be amplified as it passes through a transient space between the diaphragm and the bottom plate, and the amplified heartbeat sound may be input into the stethoscope sensor through the first sensing hole.

The diaphragm may be formed in a shape and a material that further amplify a wavelength region representing the heartbeat of the subject.

The sensing apparatus may further comprise a sealing pad disposed between the circuit board and the bottom plate and having a second sensing hole formed at a position corresponding to the first sensing hole to have a diameter smaller than that of the first sensing hole.

The circuit board may further include: a counting circuit for calculating a breathing rate using a measurement signal of the strain sensor and calculating a heartbeat rate using a measurement signal of the stethoscope sensor; and a communication circuit for transmitting at least one among the calculated breathing rate and heartbeat rate to an outside.

The sensing apparatus may further comprise an external ECG module including a plurality of electrocardiogram sensors, wherein the circuit board further includes an interface circuit for being connected with the external ECG module.

The circuit board further includes a diagnosis circuit for determining an abnormal state or a disease name of the subject using at least one among the calculated breathing rate and heart rate and the measured electrocardiogram.

According to another aspect of the present invention, there is provided a wearable diagnosis apparatus comprising: a sensing apparatus including a main body housing provided with a first coupling member and a second coupling member for coupling a strap on opposing sides, and having a pressing hole formed on a bottom surface, a strain sensor attached to the first coupling member or an inside of a first side wall of the main body housing provided with the first coupling member, and measuring a change in a shape of the first coupling member or an attached point of the first coupling member generated by tension of the strap according to breathing of the subject, a diaphragm coupled to the pressing hole and provided with a flexible plate, and a first circuit board embedded in the main body housing, and provided with a stethoscope sensor that measures a heartbeat sound of the subject amplified by the diaphragm; a computing device disposed to be spaced apart from the sensing apparatus, and embedded with a second circuit board, having a counting circuit installed therein to calculate a breathing rate using a measurement signal of the strain sensor, and a battery; and a power communication line connecting the sensing apparatus and the computing device.

The first coupling member may include: an elastic body to which the strain sensor is attached on one side; and a coupling ring to which the strap is coupled, of which one side is connected to the other side of the elastic body, and the other side is separated from the elastic body.

A mounting groove may be formed on a first side wall of the main body housing, and the first coupling member may be mounted on the main body housing through the mounting groove, wherein one side of the coupling ring coupled to the elastic body may be inserted into the mounting groove.

A bottom plate having a first sensing hole formed thereon may be further disposed at a bottom of the main body housing, the stethoscope sensor may be installed at a position corresponding to the first sensing hole on the first circuit board, the heartbeat sound transferred by deformation of the diaphragm may be amplified as it passes through a transient space between the diaphragm and the bottom plate, and the amplified heartbeat sound may be input into the stethoscope sensor through the first sensing hole.

The diaphragm may be formed in a shape and a material that further amplify a wavelength region representing the heartbeat of the subject.

The diagnosis apparatus may further comprise a sealing pad disposed between the first circuit board and the bottom plate and having a second sensing hole formed at a position corresponding to the first sensing hole to have a diameter smaller than that of the first sensing hole.

The second circuit board may include: a counting circuit for calculating a breathing rate using a measurement signal of the strain sensor and calculating a heartbeat rate using a measurement signal of the stethoscope sensor; and a communication circuit for transmitting at least one among the calculated breathing rate and heartbeat rate to an outside.

The diagnosis apparatus may further comprise an external ECG module including a plurality of electrocardiogram sensors, wherein the first circuit board may further include an interface circuit for being connected with the external ECG module.

The second circuit board may further include a diagnosis circuit for determining an abnormal state or a disease name of the subject by using at least one among the calculated breathing rate and heart rate and electrocardiogram measured by an electrocardiogram sensor.

### Advantageous Effects

According to an embodiment of the present invention, since the breathing rate of a subject is measured through a sensing value that changes through the tensile force of a strap according to breathing of the subject, and the heartbeat rate of the subject is measured through heartbeat sounds amplified through a diaphragm, a wearable sensing apparatus and a diagnosis apparatus may be designed in a simple structure.

### BRIEF DESCRIPTION OF THE DRAWINGS

FIG. 1 is a perspective view showing a wearable sensing apparatus according to embodiment 1 of the present invention.

FIGS. 2 and 3 are exploded perspective views showing the wearable sensing apparatus illustrated in FIG. 1.

FIG. 4 is a cross-sectional view showing the wearable sensing apparatus illustrated in FIG. 1.

FIG. 5 is a perspective view showing a method of attaching and detaching the first coupling member illustrated in FIG. 1.

FIG. 6 is a view showing a change in the shape of a coupling member.

FIG. 7 is a perspective view showing a modified example of the first coupling member of embodiment 1.

FIG. 8 is an exploded perspective view showing a modified example of the first coupling member of embodiment 1.

FIG. 9 is a perspective view showing a strain sensor mounted on the first coupling member.

FIG. 10 is a view showing the operation of the first coupling member illustrated in FIGS. 8 and 9.

FIG. 11 is a block diagram showing the communication circuit of the wearable sensing apparatus illustrated in FIG. 1 and an external device.

FIG. 12 is a block diagram showing the configuration of a wearable sensing apparatus according to embodiment 2 of the present invention.

FIG. 13 is a block diagram showing the configuration of a wearable sensing apparatus according to embodiment 3 of the present invention.

FIG. 14 is a perspective view showing a wearable diagnosis apparatus according to embodiment 4 of the present invention.

FIG. 15 is a cross-sectional view showing the wearable sensing apparatus shown in FIG. 13.

FIG. 16 is a cross-sectional view showing the computing device shown in FIG. 13.

FIG. 17 is a schematic view showing a wearable diagnosis apparatus according to embodiment 5 of the present invention.

### BEST MODE FOR CARRYING OUT THE INVENTION

Hereinafter, several embodiments of the present invention will be described in detail using drawings. However, this is not intended to limit the present invention to any specific embodiment, and it should be understood that all transformations, equivalents, and substitutions that include the technical spirit of the present invention are included in the scope of the present invention.

In this specification, singular expressions include plural expressions unless the context clearly indicates otherwise.

When it is stated in this specification that a component “has” or “comprises” a sub-component, unless otherwise specifically stated, it is intended that the other component may be further included, rather than excluding the component.

The terms such as “ . . . Unit,” “ . . . Module,” and “Component” in this specification mean a unit that processes at least one function or operation, and may be implemented as hardware, software, or a combination of hardware and software.

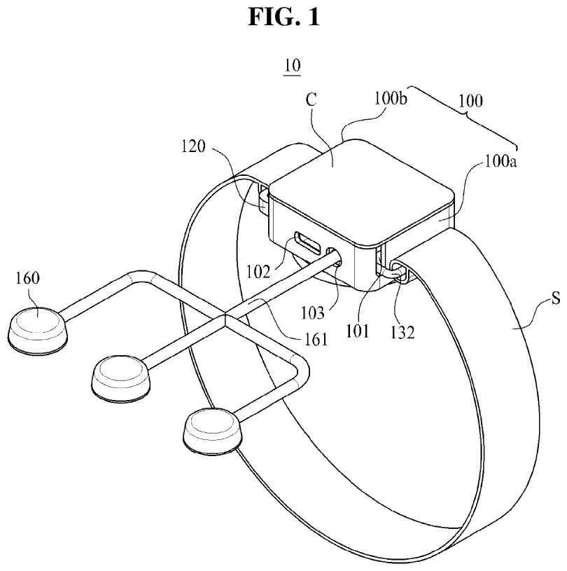

FIG. 1 is a perspective view showing a wearable sensing apparatus 10 (hereinafter, referred to as a “sensing apparatus”) according to embodiment 1 of the present invention.

The sensing apparatus 10 is worn on the body of a subject through a strap S. The sensing apparatus 10 calculates a breathing rate by measuring changes in the shape of a coupling member, which is generated by the tension of the strap S when the subject breathes. The sensing apparatus 10 measures the heartbeat rate of the subject through amplified heartbeat sounds through a diaphragm 140 that is in contact with the chest of the subject.

The strap S may be implemented in the form of a band so that it can be worn on a subject. A member such as a buckle (not shown), a snap button (not shown), or Velcro (not shown) may be provided at both end portions of the strap S to facilitate putting on and taking off of the strap S. As the strap S is implemented to have elasticity or to be able to adjust the length, it may provide a comfortable sense of fit according to the body shape of the subject.

An extended ECG (electrocardiogram) module 160 may be further connected to the sensing apparatus 10 as an option.

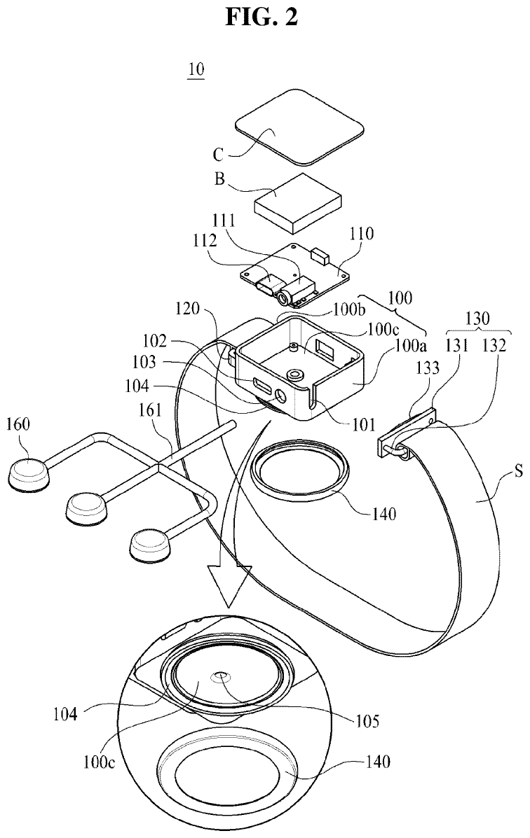

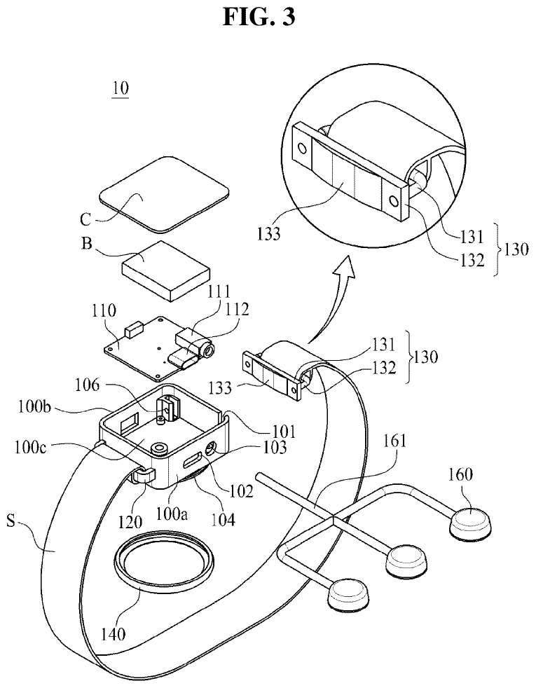

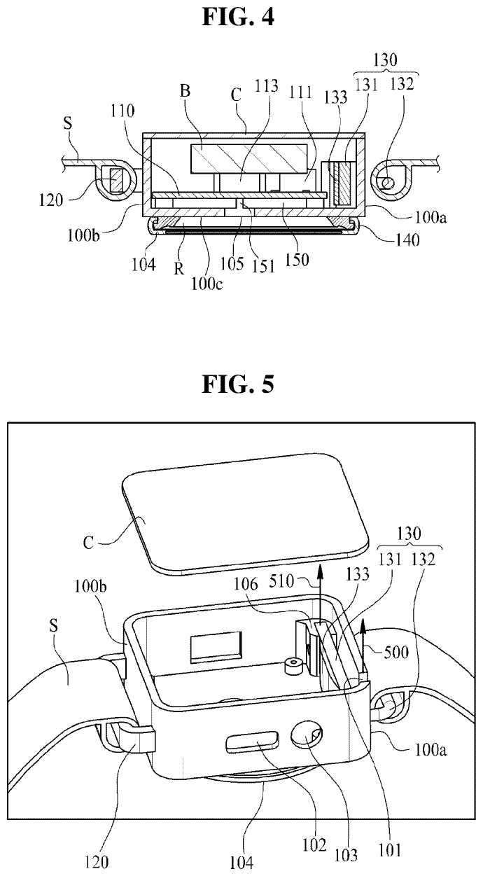

FIGS. 2 and 3 are exploded perspective views showing the sensing apparatus 10 shown in FIG. 1, and FIG. 4 is a cross-sectional view showing the sensing apparatus 10 illustrated in FIG. 1.

The main body housing 100 is a case that forms the outer shape of the sensing apparatus 10, and may be formed in a square shape or may be formed in a circular or other shape. The main body housing 100 is worn on the body of the subject through the strap S.

The top of the main body housing 100 is open, and the open top is closed by a cover C. The cover C may be detachably coupled to the main body housing 100. The method of coupling the cover C and the main body housing 100 is not limited to any one method.

A circuit board 110 is mounted inside the main body housing 100. The circuit board 110 is disposed on the bottom plate 100c of the main body housing 100. A pattern of an electric circuit is formed on the circuit board 110 so that a strain sensor 133, a battery B, an extended ECG module 161, a stethoscope sensor 113, and the like may be connected.

The battery B may be disposed on the top of the circuit board 110. The battery B may be charged in a wired or wireless manner. When the battery B is charged in a wireless manner, the circuit board 110 may be further provided with a wireless charging module (not shown). When the battery B is charged in a wired manner, a charging hole 102 through which a charging cable passes may be formed on one side portion of the main body housing 100. The charging cable passes through the charging hole 102 and is connected to a charging terminal 112 formed on the circuit board 110.

A mounting groove 101 that is open toward the cover C of the main body housing 100 (i.e., upward) is formed on a first side wall 100a of the main body housing 100. The mounting groove 101 is a groove for mounting a first coupling member 130 on the main body housing 100.

The first coupling member 130 and a second coupling member 120 are mounted on the main body housing 100.

The first coupling member 130 includes an elastic body 131 and a coupling ring 132.

The elastic body 131 is formed in a plate shape having elasticity. The elastic body 131 is disposed inside the main body housing 100 to face the first side wall 100a in which the mounting groove 101 is formed.

The coupling ring 132 is formed so that one end is coupled at a position corresponding to the mounting groove 101 of the elastic body 131 and the other end is spaced apart from the elastic body 131. That is, one end of the coupling ring 131 is coupled to the elastic body 131, and the other end of the coupling ring 131 is formed to extend in the length direction of the elastic body 131 along the outer surface of the first side wall 100a. Accordingly, when the elastic body 131 is disposed inside the main body housing 100, the first side wall 100a of the main body housing 100 is positioned at the spaced portion between the other end of the coupling ring 132 and the elastic body 131.

The first coupling member 130 may be mounted on the first side wall 100a of the main body housing 100 as the coupling ring 132 is inserted into the mounting groove 101 of the main body housing 100. In this state, the end portion of the strap S is wrapped around the coupling ring 132.

A strain sensor 133 is attached to the elastic body 131. The strain sensor 133 may be attached to a side opposite to the side of the elastic body 131 to which the coupling ring 132 is connected. The strain sensor 133 measures a change in the shape of the first coupling member 130 generated by the tension of the strap S according to breathing of the subject or a change in the shape of the point where the first coupling member 130 is attached.

For example, when the subject breathes, the rib cage repeatedly contracts and expands. When the rib cage of the subject expands, a tensile force is generated in the strap S, and the coupling ring 132 of the first coupling member 130 is pulled by the tensile force of the strap S. When the coupling ring 132 is pulled, a change occurs in the shape of the elastic body 131, and the strain sensor 133 measures the change of shape in the elastic body 131 and transmits it to the circuit board 110.

A fixing member 106, into which an end portion of the elastic body 131 (i.e., the opposite side of an end portion of the elastic body 131 corresponding to the mounting groove) is inserted, may be further formed inside the main body housing 100. The end portion of the elastic body 131 is inserted into the fixing member 106, and its position is fixed inside the main body housing 100.

The second coupling member 120 is mounted on a second side wall 100b of the main body housing 100 facing the first side wall 100a of the main body housing 100. The second coupling member 120 may be formed in a ring shape. Both end portions of the second coupling member 120 are coupled to the second side wall 100b of the main body housing 100. The second coupling member 120 may be formed to be spaced apart from the second side wall 100b by a predetermined distance so that the other end of the strap S may be coupled to the second coupling member 120 in a form wrapping around the second coupling member 120.

The second coupling member 120 may be formed in a shape the same as that of the first coupling member 130 and mounted on the main body housing 100 in the same manner as the first coupling member 130. At this point, a groove (not shown) the same as the mounting groove 101 of the first side wall 100a on which the first coupling member 130 is mounted may be formed on the second side wall 100b of the main body housing 100 on which the second coupling member 120 is mounted. In addition, the fixing member 106 may be formed inside the second side wall 100b of the main body housing 100 on which the second coupling member 120 is mounted.

A bottom plate 100c is provided on the bottom of the main body housing 100. A first sensing hole 105 is formed in a portion of the bottom plate 100c. The first sensing hole 105 formed in the bottom plate 100c connects the outer surface (diaphragm 140 side) of the bottom plate 100c and the inner surface (circuit board 110 side) of the bottom plate 100c with respect to the bottom plate 100c.

A coupling projection 104 is formed on the outer surface of the bottom plate 100c to couple the diaphragm 140. The end portion of the coupling projection 104 may be bent toward the side surface of the main body housing 100 so that the diaphragm 140 may be easily coupled. A pressing hole (not shown) for disposing the diaphragm 140 is formed on the bottom of the main body housing 100 by the coupling projection 104.

The diaphragm 140 is mounted on the coupling projection 104 to cover the entire bottom plate 100c of the main body housing 100. At this point, the diaphragm 140 is mounted to be spaced apart from the bottom plate 100c of the main body housing 100 by a predetermined distance. In this way, when the diaphragm 140 is mounted to be spaced apart from the bottom plate 100c of the main body housing 100 by a predetermined distance, a transient space R is formed between the bottom plate 100c of the main body housing 100 and the diaphragm 140.

When the sensing apparatus 10 is worn on a subject, the diaphragm 140 formed in the sensing apparatus 10 is in contact with the chest of the subject, and at this point, the heartbeat sound of the subject transferred by deformation of the diaphragm 140 is amplified as it passes through the transient space R between the diaphragm 140 and the bottom plate 100c. The degree of amplification of the heartbeat sound of the subject transferred by deformation of the diaphragm 140 may vary according to the shape of the transient space R or the size of the space.

The amplified heartbeat sound is input into the stethoscope sensor 113 provided on the circuit board 110 through the first sensing hole 105. The stethoscope sensor 113 may be provided at a position corresponding to the first sensing hole 105 on the circuit board 110. In addition, a transient hole (not shown) connecting the first sensing hole 105 and the stethoscope sensor 113 may be further formed on the circuit board 110. The transient hole may be formed to have a diameter the same as or smaller than the diameter of the first sensing hole 105.

The diaphragm 140 may be formed in a shape and a material that may further amplify the wavelength region representing the heartbeat of the subject. For example, the diaphragm 140 may be formed in the form of a flexible plate on the surface that is in contact with the subject. In addition, the diaphragm 140 may be formed in the form of a thin film made of a rubber material and may be formed in the shape of a convex lens or dome that is formed convexly toward the subject.

A sealing pad 150 may be further provided between the bottom plate 100c of the main body housing 100 and the circuit board 110. The sealing pad 150 is tightly attached to the bottom plate 100c of the main body housing 100 and the circuit board 110 so that the heartbeat sounds may not be leaked out. A second sensing hole 151 having a diameter smaller than that of the first sensing hole 105 is formed in the sealing pad 150 at a position corresponding to the first sensing hole 105. When a transient hole is formed in the circuit board 110, the transient hole may be formed to have a diameter the same as or smaller than the diameter of the second sensing hole 151.

The sensing apparatus 10 may amplify only the sound of a desired wavelength by adjusting the diameters of the first sensing hole 105 and the second sensing hole 151. For example, since the stethoscope sensor 113 may measure even a very small sound, noise may also be measured in addition to the heartbeat of the subject. However, only the sound of a desired wavelength is amplified by adjusting the diameters of the first sensing hole 105 and the second sensing hole 151, only the desired sound (i.e., the heartbeat sound) may be measured through the stethoscope sensor 113.

An expansion hole 103 may be further formed on the side surface of the main body housing 100 of the sensing apparatus 10. The expansion hole 103 is a hole through which an extension cable 161 for connecting the extended ECG module 160 passes toward the circuit board 110.

The extended ECG module 160 (electrocardiogram) is optionally connected to the sensing apparatus 10. The extended ECG module 160 is attached to the body of the subject, i.e., the arm, leg or abdomen of the subject, and records the activity current according to contraction of the heart as a curve.

The extended ECG module 160 may be connected to an interface circuit (not shown) provided on the circuit board 110 through the extension cable 161. At this point, the extension cable 161 is connected to an external connection terminal 111 connected to the interface circuit (not shown). When the extended ECG module 160 is wirelessly connected to the interface circuit, the external connection terminal may not be provided.

One or more electrocardiogram sensors may be provided in the extended ECG module 160, and even when there are two or more electrocardiogram sensors, they may be connected to the interface circuit provided in the circuit board 110 through one extension cable 161.

FIG. 5 is a perspective view showing a method of attaching and detaching the first coupling member 130 illustrated in FIG. 1.

As shown in FIG. 5, after detaching the cover C from the main body housing 100, the first coupling member 130 may be detached from the main body housing 100. When the coupling ring 132 is moved from the mounting groove 101 in the direction of the first arrow 500 and the other end portion of the elastic body 131 inserted into the fixing member 106 is moved in the direction of the second arrow 510, the first coupling member 130 is detached from the main body housing 100. In this way, according to the present invention, as the first coupling member 130 is easily detached from the main body housing 100, replacement of the strain sensor 133 according to the lifespan is easy.

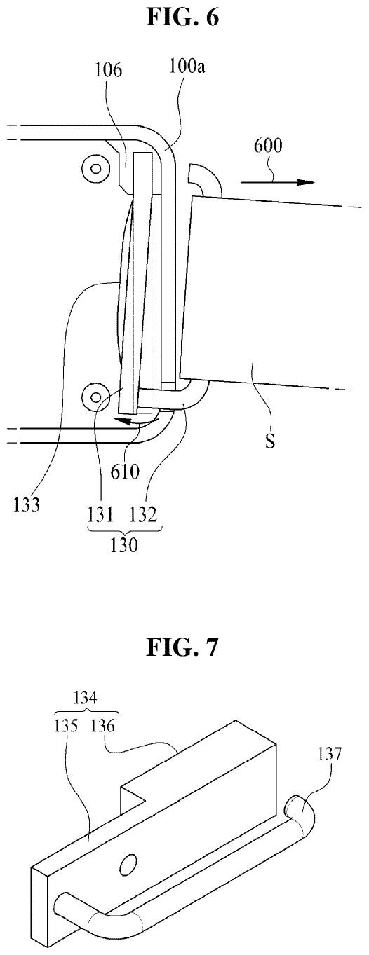

FIG. 6 is a view showing a change in the shape of the first coupling member 130. As shown in FIG. 6, when the subject breathes and the rib cage expands, a tensile force is generated in the strap S in the direction of the first arrow 600. At this point, the coupling ring 132 is pulled by the strap S, and the elastic body 131 is bent in the direction of the second arrow 610 while the other end of the elastic body 131 is fixed to the fixing member 106. The strain sensor 133 measures the displacement value for the change in the shape of the bent elastic body 131. When the subject breathes and the rib cage contracts, the elastic body 131 is restored to its original state.

FIG. 7 is a perspective view showing a modified example of the first coupling member of embodiment 1. As shown in FIG. 7, the elastic body 134 of the first coupling member 130 includes a first bulkhead unit 135 and a second bulkhead unit 136.

The first bulkhead unit 135 is a part to which the coupling ring 137 is connected. The first bulkhead unit 135 is formed so that the thickness is relatively smaller than the thickness of the second bulkhead unit 136. A strain sensor (not shown) is attached to the inner surface of the first bulkhead unit 135.

The second bulkhead unit 136 is a part inserted into a fixing member (not shown) of the main body housing (not shown). The second bulkhead unit 136 is formed to be relatively thicker than the first bulkhead unit 135.

When a tensile force is generated in the strap (not shown), a change in the shape occurs in the first bulkhead unit 135 having a relatively thin thickness, and the strain sensor measures the displacement of the first bulkhead unit 135.

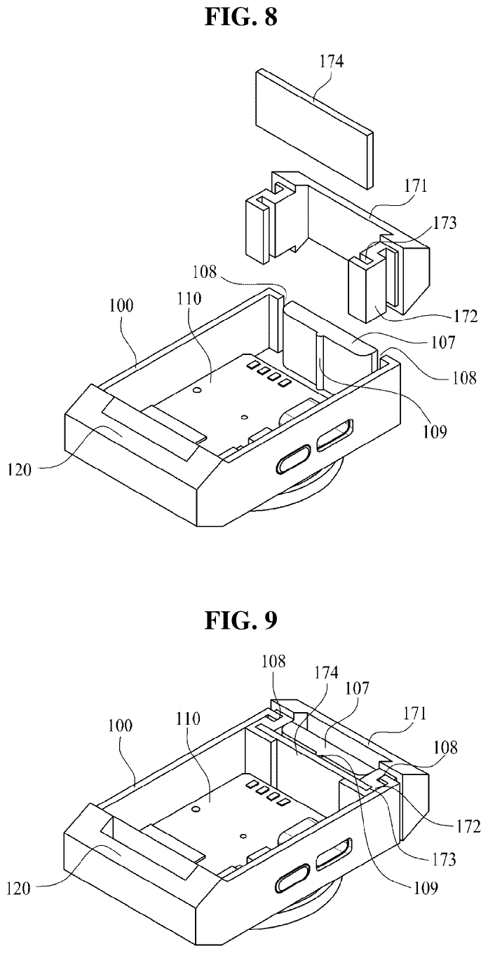

FIG. 8 is an exploded perspective view showing a modified example of the main body housing and the first coupling member of embodiment 1, and FIG. 9 is a perspective view showing a strain sensor mounted on the first coupling member shown in FIG. 8.

Referring to FIG. 8, mounting grooves 108 are formed at both end portions of the side wall 107 of the main body housing 100 on which the first coupling member 170 is formed. The top of the mounting grooves 108 is open so that the first coupling member 170 may be attached to and detached from the main body housing 100 in a sliding manner through the mounting grooves 108.

The first coupling member 170 includes a strap coupling plate 171, sensor coupling units 172, and sensor insertion grooves 173.

The strap coupling plate 171 is a part where the strap is wound. The strap coupling plate 171 is disposed so that both end portions correspond to the mounting grooves 108 formed at both end portions of the main body housing 100. For example, the strap coupling plate 171 may be formed to have a length greater than the length between the mounting grooves 108 formed at both end portions of the main body housing 100.

The sensor coupling units 172 are formed to protrude from the areas in the strap coupling plate 171 corresponding to the mounting grooves 108 toward the inside of the main body housing 100. The end portion of each sensor coupling unit 172 is bent in the shape of ‘E’. The open portion in the shape of ‘E’ defines a sensor insertion groove 173. The sensor coupling units 172 may be disposed so that the sensor fitting grooves 173 may face each other.

The end portions of the strain sensor 174 are inserted into the sensor insertion grooves 173. For example, when the strain sensor 174 is inserted into the sensor insertion grooves 173, the strain sensor 174 is disposed to be spaced apart from the inside of the side surface 107 of the main body housing 100 by a predetermined distance.

A protrusion 109 is formed inside the side wall 107 of the main body housing 100. The protrusion 109 is in contact with the strain sensor 174 mounted on the first coupling member 170. Although two or more protrusions 109 may be formed, it may be preferable to form one protrusion at a location corresponding to the center of the strain sensor 174.

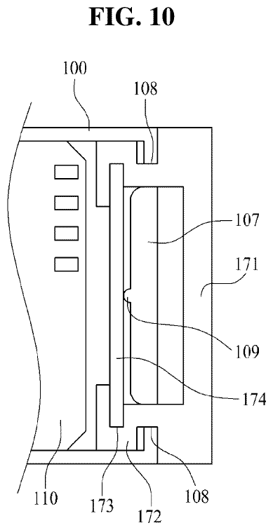

FIG. 10 is a view showing the operation of the first coupling member illustrated in FIGS. 8 and 9.

When the rib cage expands during the breathing of the subject and a tensile force (not shown) is generated in the strap, the strap coupling plate 171 of the first coupling member 170 is pulled in the direction of the tensile force of the strap, and presses the strain sensor 174 toward the side wall 107 of the main body housing 100. The strain sensor 174 is pulled toward the inner wall 107 of the main body housing 100 by the strap and comes to be in contact with the protrusion 109 so that pressure is applied to a position corresponding to the protrusion 109. The pressure value measured by the strain sensor 174 is used for measuring breathing of the subject.

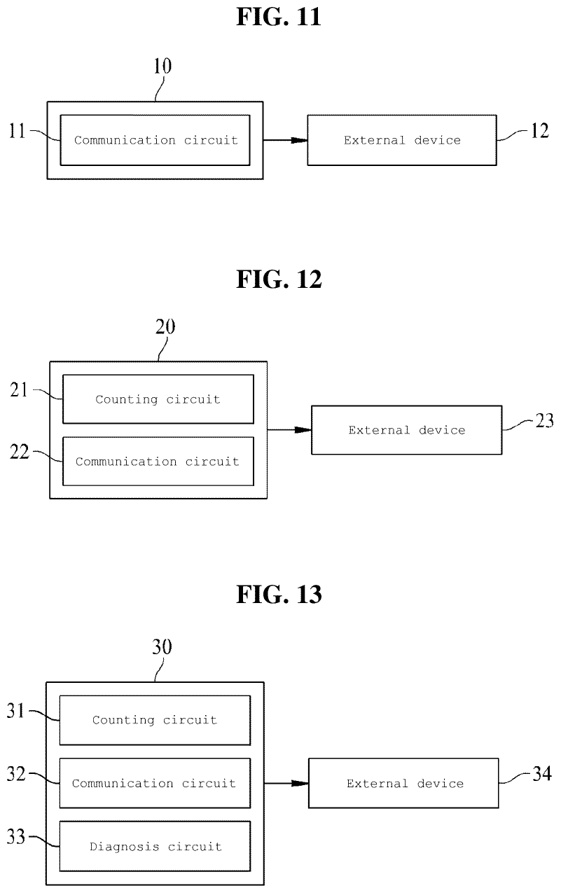

The sensing apparatus 10 of embodiment 1 shown in FIG. 1 may further include a communication circuit 11 that may be connected to an external device 12, as shown in FIG. 11.

The communication circuit 11 is provided on the circuit board 110. The communication circuit 11 transmits measurement signals of the strain sensor 133 and measurement signals of the stethoscope sensor 113 to the external device 12. Here, the external device 12 may mean a diagnosis apparatus (not shown) or an analysis apparatus (not shown). The diagnosis apparatus or the analysis apparatus receives at least one waveform signal among a heartbeat waveform signal (a measurement signal of the stethoscope sensor 113) and a respiration waveform signal (a measurement signal of the strain sensor) from the sensing apparatus 10, and may calculate at least one among the heartbeat rate or the breathing rate of the subject through the received waveform signal. In addition, the diagnosis apparatus or the analysis apparatus may also determine an abnormal state of the subject through the heartbeat rate, breathing rate, or electrocardiogram of the subject.

Meanwhile, the sensing apparatus 10 may be further provided with a temperature sensor (not shown). The temperature sensor measures external temperature of the sensing apparatus 10. Here, the external temperature means the temperature of an examination room where examinations on the subject are performed. The communication circuit 11 provides the temperature measured by the temperature sensor to the diagnosis apparatus or the analysis apparatus. The diagnosis apparatus or the analysis apparatus determines an abnormal state of the subject by comprehensively considering at least one among the heartbeat rate and the breathing rate of the subject and the external temperature measured by the temperature sensor.

For example, when at least one among the heartbeat rate and the breathing rate of the subject increases or decreases, the diagnosis apparatus or the analysis apparatus determines whether it is due to an abnormal state (e.g., a disease) or the external temperature. When at least one among the heartbeat rate and the breathing rate of the subject increases or decreases even though the external temperature is within an appropriate temperature range (room temperature), the diagnosis apparatus or the analysis apparatus determines that an abnormal state has occurred in the subject. When the external temperature is out of the appropriate temperature range (room temperature), the diagnosis apparatus or the analysis apparatus determines that increase or decrease in at least one among the heartbeat rate and the breathing rate of the subject is due to the external temperature.

Here, the appropriate temperature range (room temperature) that does not seriously affect determination of the abnormal state of the subject may be between 22 and 25°, and the heartbeat rate or the breathing rate of the subject that increases every time the temperature increases or decreases by 1° in the temperature range may be confirmed from an algorithm or a DB constructed in advance.

FIG. 12 is a block diagram showing the configuration of a wearable sensing apparatus according to embodiment 2 of the present invention. The sensing apparatus 20 of embodiment 2 may be implemented in a form the same as that of the sensing apparatus 10 of embodiment 1, and further includes a counting circuit 21 on a circuit board (not shown). As all the other configurations excluding the counting circuit 21 are the same as those of embodiment 1, duplicate description thereof will be omitted.

Respiration of a subject is divided into inspiration and expiration. During the inspiration process, as the intercostal muscles contract, the ribs are lifted, and the diaphragm also contracts and descends, the volume of the thoracic cage increases. On the other hand, during the expiration process, as the intercostal muscles relax, the ribs are lowered, and the diaphragm relaxes and rises, the volume of the thoracic cage decreases.

In the present invention, breathing may be defined in a broad sense of detecting inspiration and expiration. Or, it may mean expansion and contraction of the rib cage in a narrow sense, and may mean expansion and contraction of the lungs in a narrower sense. That is, definition of breathing in the present invention is interpreted as a meaning that includes expansion and contraction of the rib cage or lungs, as well as inspiration and expiration.

Here, when a subject breathes, the rib cage expands, and the moment when the rib cage expands to the maximum is the moment when the breathing changes, i.e., the moment when the breathing changes from inhalation to exhalation. When the rib cage expands during the inhalation of the subject, a tensile force is generated in the strap S. At this point, the coupling ring is pulled by the strap, and the elastic body is bent while the end portion of the elastic body is fixed to the fixing member. In addition, when the rib cage contracts during the exhalation of breathing of the subject, the elastic body returns to its original state. The strain sensor measures the displacement value for the change in the shape of the bent elastic body.

The counting circuit 21 calculates a breathing rate per minute using a peak value and the next peak value of the respiration waveform measured by the strain sensor.

The heartbeat sound transferred by deformation of the diaphragm is amplified as it passes through the transient space R between the diaphragm and the bottom plate, and the amplified heartbeat sound is measured by the stethoscope sensor 113 through the first sensing hole formed on the bottom plate of the main body housing 100.

The counting circuit 21 calculates a heartbeat rate per minute using a peak value and the next peak value of the heartbeat waveform measured by the stethoscope sensor 113.

The communication circuit 22 transmits at least one among the breathing rate and the heartbeat rate calculated by the counting circuit 21 to the external device 23. The external device 23 may be a diagnosis apparatus or an analysis apparatus, and the diagnosis apparatus or the analysis apparatus may also determine an abnormal state of the subject through the heartbeat rate, breathing rate, or electrocardiogram of the subject.

Meanwhile, the sensing apparatus 20 may be further provided with a temperature sensor (not shown). The temperature sensor measures external temperature of the sensing apparatus 20. Here, the external temperature means the temperature of an examination room where examinations on the subject are performed. The communication circuit 22 provides the external temperature measured by the temperature sensor to the external device 23 (i.e., a diagnosis apparatus or an analysis apparatus).

The diagnosis apparatus or the analysis apparatus determines an abnormal state of the subject by comprehensively considering at least one among the heartbeat rate and the breathing rate of the subject and the external temperature measured by the temperature sensor.

FIG. 13 is a block diagram showing the configuration of a wearable sensing apparatus according to embodiment 3 of the present invention. The sensing apparatus 30 of embodiment 3 may be implemented in a form the same as that of the sensing apparatus 10 of embodiment 1, and further includes a counting circuit 31 and a diagnosis circuit 33 on a circuit board. As all the other configurations excluding the counting circuit 31 and the diagnosis circuit 33 are the same as those of embodiment 1, duplicate description thereof will be omitted. In addition, as the counting circuit 31 is the same as that of embodiment 2, duplicate description thereof will be omitted.

The diagnosis circuit 33 may determine an abnormal state of the subject through the heartbeat rate or the breathing rate of the subject measured by the counting circuit 31. For example, the diagnosis circuit 33 may determine that an abnormal state has occurred in the subject when at least one among the heartbeat rate and the breathing rate of the subject increases or decreases. In addition, the diagnosis circuit 33 may determine that an abnormal state has occurred when the heartbeat rate or the breathing rate of the subject is irregular.

Meanwhile, the sensing apparatus 30 may be further provided with a temperature sensor (not shown). The temperature sensor measures external temperature of the sensing apparatus 30. Here, the external temperature means the temperature of an examination room where examinations on the subject are performed. The diagnosis circuit 33 determines an abnormal state of the subject by comprehensively considering at least one among the heartbeat rate and the breathing rate of the subject and the external temperature measured by the temperature sensor.

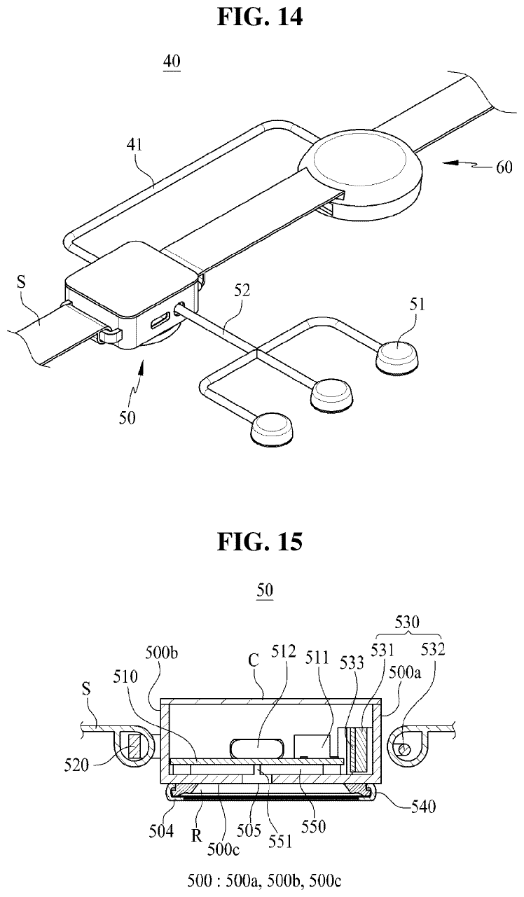

FIG. 14 is a perspective view showing a wearable diagnosis apparatus according to embodiment 4 of the present invention.

The wearable diagnosis apparatus 40 of embodiment 4 (hereinafter, referred to as the “diagnosis apparatus”) separates the circuits for measuring the breathing rate or heartbeat rate through the signals measured by the sensors from the devices related to sensors in embodiment 1. The components related to the sensors are embedded in the sensing apparatus 50, and the circuits for measuring the breathing rate or heartbeat rate are embedded in a computing device 60.

For example, when the subject is a small animal, the size of the sensing apparatus 50 is limited. When a large sensing apparatus 50 is used despite a small animal, a measurement error may occur. For example, in the case of a small dog breed, the shape of the chest is formed in a shape sharply protruding toward the front, and when the sensing apparatus 50 is formed large, it may not be completely attached to the chest of the small dog breed, and a measurement error may occur.

According to embodiment 4, the size of the sensing apparatus 10 may be designed small by embedding the devices related to sensors in the sensing apparatus 50 and separating the other components in the computing device 60.

FIG. 15 is a cross-sectional view showing the sensing apparatus shown in FIG. 14. Referring to FIGS. 14 and 15, the diagnosis apparatus 40 of embodiment 4 includes a sensing apparatus 50, a computing device 60, and a power communication line 41.

Components related to sensors are embedded in the sensing apparatus 50.

Comparing the sensing apparatus 50 shown in FIG. 15 with the sensing apparatus 10 of embodiment 1, since only the drawing numerals of each component are different, and the functions are the same, except that the battery B is not included in the main body housing 100, but is included in the computing device 60, and a communication through hole (not shown) for connection of the power communication line 41 is formed in the side wall of the main body housing 500, only the components of the sensing apparatus 50 of embodiment 5 that are different from those of the sensing apparatus 10 of embodiment 1 will be described hereinafter for convenience of explanation.

First, a communication through hole (not shown) through which the power communication line 41 passes is formed on the side surface of the main body housing 500 of the sensing apparatus 50. The power communication line 41 is connected to the communication terminal of the first circuit board 510 through the communication through hole.

The first circuit board 510 is installed inside the main body housing 500. Since the functions of the first circuit board 510 are the same as those of the circuit board 100, except that the first circuit board 510 has an electric circuit pattern formed for connection to the power communication line 41, instead of an electric circuit pattern for connection to the battery B, as the sensing apparatus 50 does not include the battery B, detailed description thereof will be omitted.

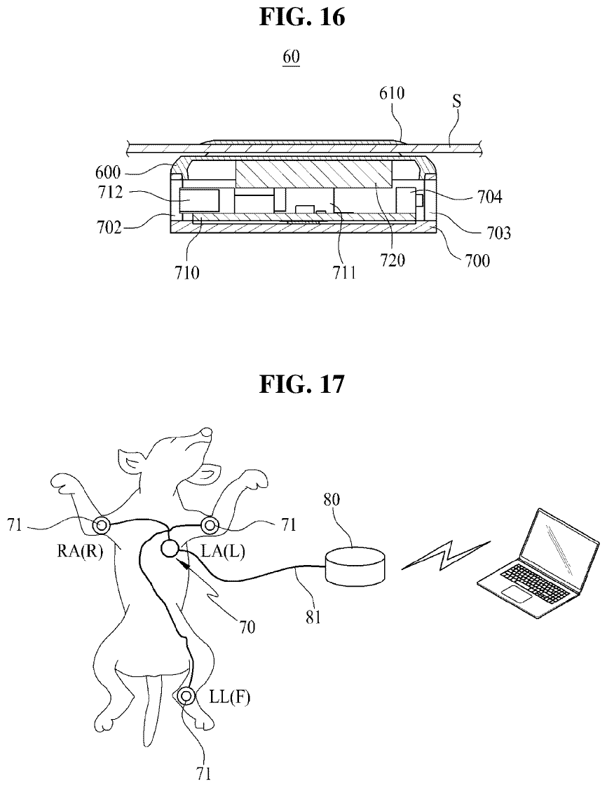

FIG. 16 is a cross-sectional view showing the computing device shown in FIG. 14.

Referring to FIGS. 14 and 16, the computing device 60 is disposed to be spaced apart from the sensing apparatus 50 while being coupled to the strap S. The computing device 60 is embedded with a battery B that supplies power to the sensing apparatus 50, a counting circuit that measures the breathing rate or heartbeat rate through measurement signals of the sensing apparatus 50, and the like.

The computing device 60 includes an upper case 600 and a lower case 700.

A pair of coupling holes 610 for coupling the strap S are formed on the side surfaces of the upper case 600 of the computing device 60. The strap S passes through the inside of the upper case 600 through the pair of coupling holes 610. Although it is shown that the top of the upper case 600 is formed closed so that the strap S is not visible, it may be formed open so that the strap S is visible.

The lower case 700 of the computing device 60 is coupled to the bottom of the upper case 600. A second circuit board 710 is installed inside the lower case 700 of the computing device 60.

A pattern of an electric circuit is formed on the second circuit board 710 so that a battery B and a counting circuit (not shown) may be connected.

A charging hole 702 may be formed on the side surface of the lower case 700. The charging hole 702 is a hole through which a charging cable for charging the sensing apparatus 50 passes.

A communication through hole (not shown) is formed on the side surface of the lower case 700 of the computing device 60 through which the power communication line 41 passes. The power communication line 41 is connected to the communication terminal (not shown) of the second circuit board 710 through the communication through hole.

The battery B may supply power to the first circuit board 510 through the power communication line 41. The battery B may be charged in a wired or wireless manner, and when the battery B is charged in a wireless manner, a wireless charging module may be further provided on the second circuit board 710. When the battery B is charged in a wired manner, a charging hole 702 through which the charging cable passes may be formed on the side surface of the lower case 700 of the computing device 60. The charging cable passes through the charging hole 702 and is connected to a charging terminal 712 formed on the circuit board 710.

The counting circuit calculates a breathing rate using the measurement signal of the strain sensor (533 in FIG. 15). The counting circuit calculates a breathing rate per minute using a peak value and the next peak value of the respiration waveform measured by the strain sensor. The counting circuit calculates a heartbeat rate per minute using a peak value and the next peak value of the heartbeat waveform measured by the stethoscope sensor (512 in FIG. 15).

The communication circuit (not shown) transmits at least one among the breathing rate and the heartbeat rate calculated by the counting circuit to an external device. The external device may be a diagnosis apparatus or an analysis apparatus, and the diagnosis apparatus or the analysis apparatus may also determine an abnormal state of the subject through the heartbeat rate or the breathing rate of the subject.

Meanwhile, the second circuit board 710 of the computing device 60 may further include a diagnosis circuit (not shown).

The diagnosis circuit may determine an abnormal state of the subject through the heartbeat rate, breathing rate, or electrocardiogram of the subject measured by the counting circuit. For example, the diagnosis circuit may determine that an abnormal state has occurred in the subject when at least one among the heartbeat rate and the breathing rate of the subject increases or decreases. In addition, the diagnosis circuit may determine that an abnormal state has occurred when the heartbeat rate or the breathing rate of the subject is irregular.

The communication circuit transmits the information determined by the diagnosis circuit to an external device.

One among the computing device 60 and the sensing apparatus 50 may be further provided with a temperature sensor (not shown) for sensing the external temperature. Here, the external temperature means the temperature of an examination room where examinations on the subject are performed. The diagnosis circuit determines an abnormal state of the subject by comprehensively considering at least one among the heartbeat rate and the breathing rate of the subject and the external temperature measured by the temperature sensor. Even when the diagnosis circuit is not provided, the temperature sensor may be provided on the second circuit board 710, and at this point, the measurement value of the temperature sensor may be transmitted to an external device by a communication circuit (not shown).

FIG. 17 is a schematic view showing a diagnosis apparatus of embodiment 5 of the present invention.

As the sensing apparatus 70 and the extended ECG module 71 of embodiment 5 are the same as the sensing apparatus 50 and the extended ECG module 51 of embodiment 4, duplicate description thereof will be omitted.

The computing device 80 is connected to the sensing apparatus 70 through the power communication line 81. The computing device 80 includes a second circuit board, in which a counting circuit for calculating the breathing rate using the measurement signal of the strain sensor is installed, and a battery. Since the counting circuit, the second circuit board, and the battery are the same as the counting circuit, the second circuit board 710, and the battery B of embodiment 4, duplicate description thereof will be omitted. The external appearance of the computing device 80 may be formed in any shape as long as the counting circuit, the second circuit board, and the battery are embedded. The computing device 80 may include a communication circuit for transmitting the breathing rate calculated by the counting circuit to the outside.

The communication circuit transmits at least one among the breathing rate and the heartbeat rate calculated by the counting circuit to an external device. The external device may be a diagnosis apparatus or an analysis apparatus, and the diagnosis apparatus or the analysis apparatus may determine an abnormal state of the subject through the heartbeat rate, breathing rate, or electrocardiogram of the subject. The communication circuit may support at least one among wireless communication and wired communication. The computing device 60 may be selectively connected to the external device through the wireless or wired communication.

The computing device 80 may further include a diagnosis circuit. The diagnosis circuit may determine an abnormal state of the subject through the heartbeat rate or the breathing rate of the subject measured by the counting circuit. For example, the diagnosis circuit may determine that an abnormal state has occurred in the subject when at least one among the heartbeat rate and the breathing rate of the subject increases or decreases. In addition, the diagnosis circuit may determine that an abnormal state has occurred when the heartbeat rate or the breathing rate of the subject is irregular. The communication circuit transmits the result of the diagnosis circuit to the external device.

Although the present invention has been described above with reference to several embodiments, it will be understood by those skilled in the art that the present invention may be modified and changed in various ways without departing from the spirit and scope of the present invention described below in the claims.

In addition, some functions of the apparatus or system described above may be provided to be included in a computer-readable recording medium, as a program of commands for implementing the same is tangibly implemented. The computer-readable recording medium may include program commands, data files, data structures, and the like separately or in combination. Examples of the computer-readable recording medium include magnetic media such as hard disks, floppy disks, and magnetic tapes, optical media such as CD-ROMs and DVDs, magneto-optical media such as floptical disks, and hardware devices specifically configured to store and execute program commands, such as ROMs, RAMs, flash memories, and USB memories.

1. A wearable sensing apparatus comprising:
a main body housing provided with a first coupling member and a second coupling member for coupling a strap on opposing sides, and having a pressing hole formed on a bottom surface;
a strain sensor attached to the first coupling member or an inside of a first side wall of the main body housing provided with the first coupling member, and measuring a change in a shape of the first coupling member or an attached point of the first coupling member generated by tension of the strap according to breathing of the subject;
a diaphragm coupled to the pressing hole and provided with a flexible plate; and
a circuit board embedded in the main body housing, and provided with a stethoscope sensor that measures a heartbeat sound of the subject amplified by the diaphragm.

2. The sensing apparatus according to claim 1, wherein the first coupling member includes:
an elastic body to which the strain sensor is attached on one side; and
a coupling ring to which the strap is coupled, of which one side is connected to the other side of the elastic body, and the other side is separated from the elastic body.

3. The sensing apparatus according to claim 2, wherein a mounting groove is formed on a first side wall of the main body housing, and the first coupling member is mounted on the main body housing through the mounting groove, wherein
one side of the coupling ring coupled to the elastic body is inserted into the mounting groove.

4. The sensing apparatus according to claim 1, wherein a bottom plate having a first sensing hole formed thereon is further disposed at a bottom of the main body housing, the stethoscope sensor is installed at a position corresponding to the first sensing hole on the circuit board, the heartbeat sound transferred by deformation of the diaphragm is amplified as it passes through a transient space between the diaphragm and the bottom plate, and the amplified heartbeat sound is input into the stethoscope sensor through the first sensing hole.

5. The sensing apparatus according to claim 4, further comprising a sealing pad disposed between the circuit board and the bottom plate and having a second sensing hole formed at a position corresponding to the first sensing hole to have a diameter smaller than that of the first sensing hole.

6. The sensing apparatus according to claim 1, wherein the diaphragm is formed in a shape and a material that further amplify a wavelength region representing the heartbeat of the subject.

7. The sensing apparatus according to claim 1, wherein the circuit board further includes:
a counting circuit for calculating a breathing rate using a measurement signal of the strain sensor and calculating a heartbeat rate using a measurement signal of the stethoscope sensor; and
a communication circuit for transmitting at least one among the calculated breathing rate and heartbeat rate to an outside.

8. The sensing apparatus according to claim 7, wherein the circuit board further includes a diagnosis circuit for determining an abnormal state or a disease name of the subject by using at least one among the calculated breathing rate and heart rate and electrocardiogram measured by an electrocardiogram sensor.

9. The sensing apparatus according to claim 1, further comprising an external ECG module including a plurality of electrocardiogram sensors, wherein the circuit board further includes an interface circuit for being connected with the external ECG module.

10. A wearable diagnosis apparatus comprising:
a sensing apparatus including a main body housing provided with a first coupling member and a second coupling member for coupling a strap on opposing sides, and having a pressing hole formed on a bottom surface, a strain sensor attached to the first coupling member or an inside of a first side wall of the main body housing provided with the first coupling member, and measuring a change in a shape of the first coupling member or an attached point of the first coupling member generated by tension of the strap according to breathing of the subject, a diaphragm coupled to the pressing hole and provided with a flexible plate, and a first circuit board embedded in the main body housing, and provided with a stethoscope sensor that measures a heartbeat sound of the subject amplified by the diaphragm;
a computing device disposed to be spaced apart from the sensing apparatus, and embedded with a second circuit board, having a counting circuit installed therein to calculate a breathing rate using a measurement signal of the strain sensor, and a battery; and
a power communication line connecting the sensing apparatus and the computing device.

11. The diagnosis apparatus according to claim 10, wherein the first coupling member includes:
an elastic body to which the strain sensor is attached on one side; and
a coupling ring to which the strap is coupled, of which one side is connected to the other side of the elastic body, and the other side is separated from the elastic body.

12. The diagnosis apparatus according to claim 11, wherein a mounting groove is formed on a first side wall of the main body housing, and the first coupling member is mounted on the main body housing through the mounting groove, wherein
one side of the coupling ring coupled to the elastic body is inserted into the mounting groove.

13. The diagnosis apparatus according to claim 10, wherein a bottom plate having a first sensing hole formed thereon is further disposed at a bottom of the main body housing, the stethoscope sensor is installed at a position corresponding to the first sensing hole on the first circuit board, the heartbeat sound transferred by deformation of the diaphragm is amplified as it passes through a transient space between the diaphragm and the bottom plate, and the amplified heartbeat sound is input into the stethoscope sensor through the first sensing hole.

14. The diagnosis apparatus according to claim 13, further comprising a sealing pad disposed between the first circuit board and the bottom plate and having a second sensing hole formed at a position corresponding to the first sensing hole to have a diameter smaller than that of the first sensing hole.

15. The diagnosis apparatus according to claim 10, wherein the diaphragm is formed in a shape and a material that further amplify a wavelength region representing the heartbeat of the subject.

16. The diagnosis apparatus according to claim 10, wherein the second circuit board includes:
a counting circuit for calculating a breathing rate using a measurement signal of the strain sensor and calculating a heartbeat rate using a measurement signal of the stethoscope sensor; and
a communication circuit for transmitting at least one among the calculated breathing rate and heartbeat rate to an outside.

17. The diagnosis apparatus according to claim 16, wherein the second circuit board further includes a diagnosis circuit for determining an abnormal state or a disease name of the subject by using at least one among the calculated breathing rate and heart rate and electrocardiogram measured by an electrocardiogram sensor.

18. The diagnosis apparatus according to claim 10, further comprising an external ECG module including a plurality of electrocardiogram sensors, wherein the first circuit board further includes an interface circuit for being connected with the external ECG module.
